(19) United States
(12) Patent Application Publication
(10) Pub. No.: US 2006/0085003 A1
(43) Pub. Date: Apr. 20, 2006

(54) Use of autogenous growth factors in bone tunnels during ligament reconstruction

(75) Inventors: Reinhold Schmieding, Naples, FL (US); Matthew C. Summitt, Naples, FL (US)

Correspondence Address: DICKSTEIN SHAPIRO MORIN & OSHINSKY LLP, 2101 L Street, NW, Washington, DC, 20037, US

(73) Assignee: Arthrex, Inc.

(21) Appl. No.: 11/240,392

(22) Filed: Oct. 3, 2005

Related U.S. Application Data
(60) Provisional application No. 60/615,592, filed on Oct. 5, 2004.

(51) Int. Cl. A61B 17/58

(52) U.S. Cl. 606/73

(57) ABSTRACT

A method for introducing autogenous growth factors produced from a patient's own blood into a target bone tunnel created by the surgeon during ligament reconstruction. The autogenous growth factors, preferably contained in platelet-rich plasma, are provided optionally with hyaluronic acid and with a coagulating agent such as thrombin to form a coagulated or viscous PRP. The coagulated growth factors are inserted into the target tunnel, to enhance the healing of the implant or graft and to promote bone growth.

This application claims the benefit of U.S. Provisional Application Ser. No. 60/615,592, filed on Oct. 5, 2004, the entire disclosure of which is incorporated by reference herein.

### FIELD OF THE INVENTION

The present invention relates to methods of reconstruction surgery and, in particular, to a method of ligament reconstruction using autogenous growth factors.

### BACKGROUND OF THE INVENTION

Methods of ligament reconstruction in the knee are known in the art. Reconstruction of anterior cruciate ligament, for example, is described in U.S. Pat. Nos. 5,211,647 and 5,320,626 and typically involves drilling a tunnel through the tibia, drilling a closed tunnel (socket) into the femur, inserting a substitute graft from one tunnel to the other, and securing the respective ends of the graft to the walls of the tibial and femoral tunnels using interference screws or transverse pins.

Although the existing method of ligament reconstruction described above is now widely practiced, it nevertheless has a number of attendant disadvantages. For example, extensive transosseous tunnels may be created to position replacement grafts in anatomical positions to reproduce the function of the damaged or absent ligament. Biologic incorporation of the replacement graft in the tunnel and the subsequent healing of the tunnel require an extensive reduction of immobilization, reduced patient activity levels, loss of motion and delayed return to normal daily functions during the lengthy healing process. As a result, post-operatory loosening or lengthening of the graft may also occur.

### SUMMARY OF THE INVENTION

The present invention provides a method for introducing autogenous growth factors produced from a patient's own blood into a target bone tunnel created by the surgeon during ligament reconstruction. The autogenous growth factors may be coagulated with a coagulant such as thrombin or mixed with a viscous substance such as hyaluronic acid before being introduced into the target bone tunnel. Alternatively, a graft tendon is soaked in autogenous growth factors, preferably soaking the graft in platelet-rich plasma (referred to hereinafter as “PRP”), optionally with hyaluronic acid added. The soaked graft is placed into the target bone tunnel, subsequent to which autogenous growth factors, preferably contained in PRP, may be optionally introduced into the bone tunnel and around the soaked graft. The PRP significantly reduces leakage in the bone tunnel, while enhancing the healing of the graft provided within the target bone tunnel and promoting bone growth.

The invention provides a method of conducting a ligament reconstruction employing autogenous growth factors, preferably growth factors contained in PRP, in a surgical procedure for which the growth of bone and/or tissue structure is promoted and leakage in the bone tunnel is reduced. The method comprises inserting a mixture of autogenous growth factors, preferably contained in PRP, and optionally combined with hyaluronic acid into a bone tunnel for ligament reconstruction. The PRP containing the growth factors is preferably coagulated with a coagulating agent such as thrombin to form a coagulated or viscous PRP prior to be inserted into the target tunnel. The tunnel can optionally be plugged with a synthetic plug, bone plug or screw to contain the coagulated PRP in the tunnel and accelerate healing, while also reducing leakage of the PRP and securing the graft in the tunnel.

The graft may be secured into the target tunnel by employing two cannulated interference screws, each provided at one end of the target tunnel, plugging one of the two cannulated interference screws located at one end of the tunnel, inserting the coagulated PRP into the tunnel through the cannulation of the unplugged screw, and then plugging the screw to contain the coagulated PRP in the tunnel during healing.

These and other features and advantages of the invention will be more apparent from the following detailed description that is provided in connection with the accompanying drawings and illustrated exemplary embodiments of the invention.

### BRIEF DESCRIPTION OF THE DRAWINGS

FIG. 1 illustrates a mixture of autogenous growth factors and hyaluronic acid in accordance with an embodiment of the present invention;

FIG. 2 illustrates a mixture of autogenous growth factors and a coagulant agent in accordance with another embodiment of the present invention;

FIG. 3 illustrates the injection of a mixture of autogenous growth factors and hyaluronic acid into a tibial tunnel in accordance with a method of the present invention;

FIG. 4 illustrates parts of the mixture of autogenous growth factors and hyaluronic acid introduced into the tibial tunnel but leaking into the joint space;

FIG. 5 illustrates a top view of a tibial tunnel injected with a coagulated PRP at the tip of the femoral retroscrew in accordance with a method of the present invention;

FIG. 6 illustrates a graft soaked into a mixture of autogenous growth factors in accordance with a method of the present invention; and

FIG. 7 illustrates a graft soaked into a mixture of autogenous growth factors and hyaluronic acid in accordance with a method of the present invention.

### DETAILED DESCRIPTION OF THE PREFERRED EMBODIMENT

In the following detailed description, reference is made to various specific embodiments in which the invention may be practiced. These embodiments are described with sufficient detail to enable those skilled in the art to practice the invention, and it is to be understood that other embodiments may be employed, and that structural and logical changes may be made without departing from the spirit or scope of the present invention.

The term “endoscopy” encompasses arthroscopy, laparoscopy, hysteroscopy, among others, and endoscopic surgery involves the performance of surgical procedures within a patient's body through small openings as opposed to conventional open surgery through large incisions.

The term “tunnel” or “bone tunnel” as used in the present application is intended to include any hole in bone, whether having a closed or an open end or ends, such a closed end socket or a tunnel that passes completely through the bone.

The term “growth factor” as used in the present application is intended to include all factors, such as proteinaceous factors, for example, which play a role in the induction or conduction of growth of bone, ligaments, cartilage or other tissues associated with bone or joints. In particular, these growth factors include bFGF, aFGF, EGF (epidermal growth factor), PDGF (platelet-derived growth factor), IGF (insulin-like growth factor), TGF-β I through III, including the TGF-β superfamily, VEGF, BMP 1 through 12, and GDF 1 through 12.

In a preferred embodiment, however, the term “growth factor” includes autogenous growth factors produced from a patient's own blood, preferably contained in platelet-rich plasma (PRP) obtained by a centrifugation process. In an exemplary embodiment, platelet-rich plasma (PRP) is prepared from a relatively small sample of patient's blood by multiple-step centrifugation. In this manner, at the end of the centrifugation process, an autologous platelet concentrate is obtained which is rich in growth factors. In an exemplary embodiment, the autologous platelet concentrate is introduced into a target site, for example, into a bone tunnel provided with a graft during reconstructive knee surgery. The bone tunnel can then optionally be sealed within the tunnel by fixation devices. The fixation devices prevent leaking of the autologous platelet concentrate from the target tunnel and promote the growth of bone and/or tissue structure and the overall healing process.

In an exemplary embodiment, PRP is prepared using a commercially available PRP concentration kits such as the SmartPReP® 2APC+ Platelet Concentration System sold by Harvest Technologies or the Symphony Platelet Concentration System sold by DePuy. Optionally, the resultant PRP, enriched with growth factors, may be mixed with a viscous substance, such as hyaluronic acid, to increase its viscosity and to ensure its adhesion to the target tunnel and graft.

The present invention thus provides a method for introducing autogenous growth factors produced from a patient's own blood into a target bone tunnel created by a surgeon during ligament reconstruction.

As described in more detail below, and in accordance with one embodiment of the present invention, autogenous growth factors, preferably contained in PRP, optionally with hyaluronic acid, are coagulated with a coagulant such as thrombin and provided within the target bone tunnel. The coagulated PRP may be used not only for soaking of the graft or ligament, but may also be injected into the bone tunnel subsequent to the insertion of the graft or ligament within the target bone tunnel. If the graft is secured in the tunnel with cannulated interference screws, the tunnel may then option be plugged at one end of the target tunnel, and the autogenous growth factors injected into the target tunnel through an unplugged cannulated interference screw located at the other end of the tunnel. The fixation devices and the viscosity of the autogenous growth factors prevent their leakage from the target tunnel and promote the growth of bone and/or tissue structure and the overall healing process.

In an exemplary embodiment, PRP is prepared by a centrifugation process using PRP concentration kits such as Osteokine PRP concentration kit manufactured by Orthogen GmbH of Dusseldorf, Germany. The Osteokine test kit is a disposable kit in which patient's blood is processed employing a double pouch system and a specially adapted centrifuge to obtain a PRP. The Osteokine test kit allows for the preparation of PRP from a relatively small sample of patient's body (approximately 55 ml) by multiple-steps centrifugation. In this manner, at the end of the centrifugation process, an autologous platelet concentrate is obtained free of white blood cells, red blood cells, plasma or residue. Approximately 6 ml of PRP may be obtained from each procedure. The PRP obtained was slightly more viscous than water and it was not sticky. For this reason, it was necessary to either coagulate the PRP or to mix it with a viscous substance, such as hyaluronic acid, to ensure that it is secured in the tibial tunnel.

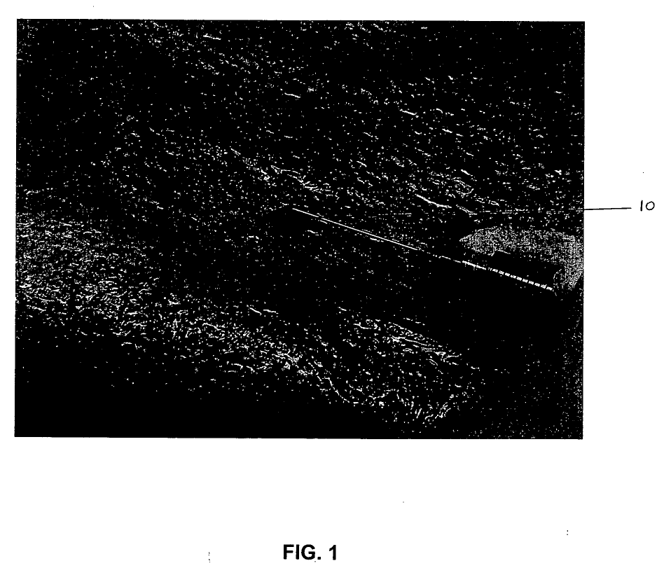

To increase the viscosity of the PRP, the PRP may be mixed with hyaluronic acid to obtain a mixture 10 (FIG. 1) of PRP and hyaluronic acid. As illustrated in FIG. 1, the addition of hyaluronic acid allows the PRP to become more viscous and to stick together when injected out of the syringe.

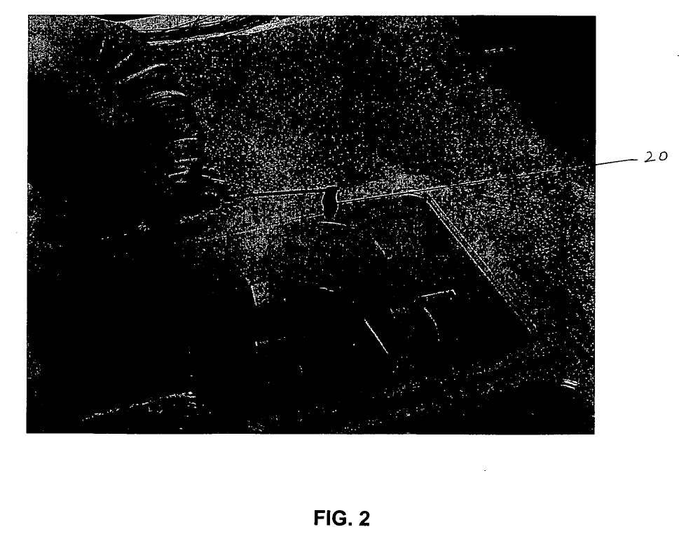

FIG. 2 illustrates the coagulation of the PRP, or of mixture 10 of PRP and hyaluronic acid of FIG. 1. At least two coagulant agents, calcium chloride and thrombin, may be employed for the coagulation to occur. When calcium chloride is added to PRP, the PRP coagulates in approximately 15 minutes. Thrombin causes a more rapid coagulation. FIG. 2 shows coagulated PRP or coagulum 20 formed after the addition of thrombin to PRP. By using a higher concentration of thrombin and mixing it more consistently more coagulum can be produced.

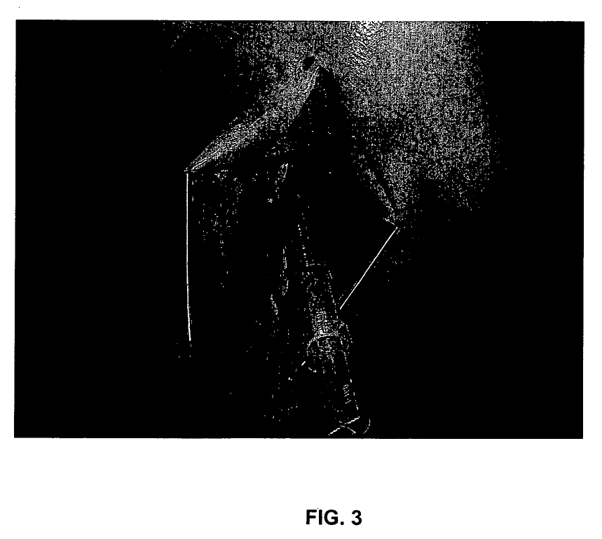

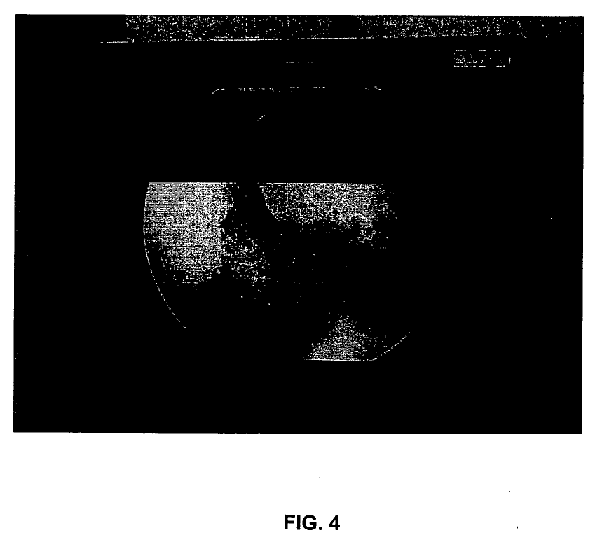

FIGS. 3-7 illustrate methods of conducting a ligament reconstruction employing autogenous growth factors, preferably PRP, optionally with hyaluronic acid, in a surgical procedure for which the growth of bone and/or tissue structure is promoted and leakage in the bone tunnel is reduced. An ACL reconstruction in the tibial tunnel was performed using the retroscrew procedure. Referring to FIG. 3 and according to one embodiment of the present invention, a tibial retroscrew was first placed in the proximal tibia to fixate the tendon. The graft was then fixated in the distal tibia with a femoral retroscrew. The tibial tunnel was then filled with a mixture of PRP and hyaluronic acid, by injecting the mixture of PRP and hyaluronic acid into the tunnel and through the cannulation of the screw, and/or injecting directly into the tendon, either post or pre insertion of the tendon. Although the mixture of PRP and hyaluronic acid promotes the growth of bone and/or tissue structure, part of the mixture of PRP and hyaluronic acid leaks into the joint space through the tibial retroscrew, as shown in FIG. 4. In addition, when the syringe is pulled back from the tibial retroscrew, additional parts of the mixture of PRP and hyaluronic acid leak into the joint space through the cannulation of the femoral retroscrew.

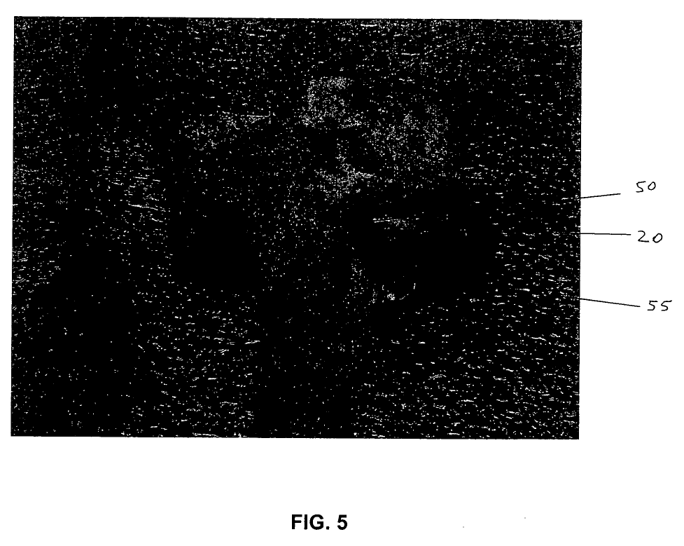

According to another embodiment of the present invention, a coagulum comprising a mixture of PRP and a coagulant (for example, coagulum 20 of FIG. 2 comprising PRP and thrombin as the coagulant, with optional hyaluronic acid) was placed into the tibial tunnel and then the femoral retroscrew was fixated in the distal tibia. FIG. 5 shows a top view of tibial tunnel 50 with the coagulated PRP 20 at the tip of femoral retroscrew 55 in the tibial tunnel 50. Although the coagulated PRP 20 is secured in the tibial tunnel 50, it is difficult to place it around the proximal aperture, which is the location where it is most needed.

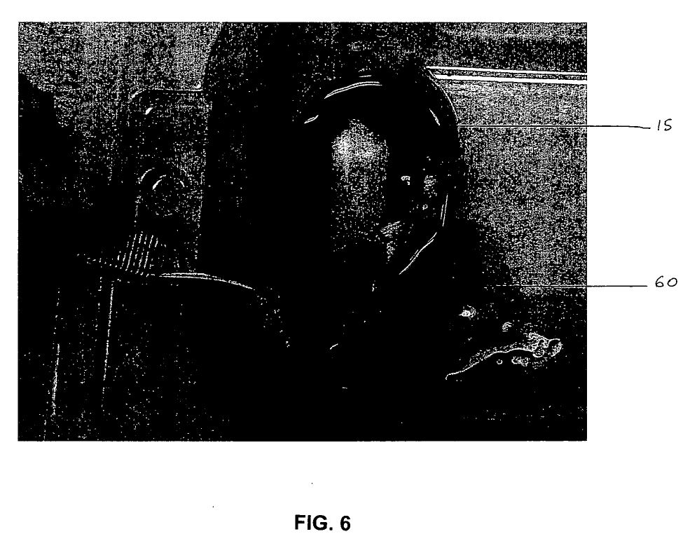

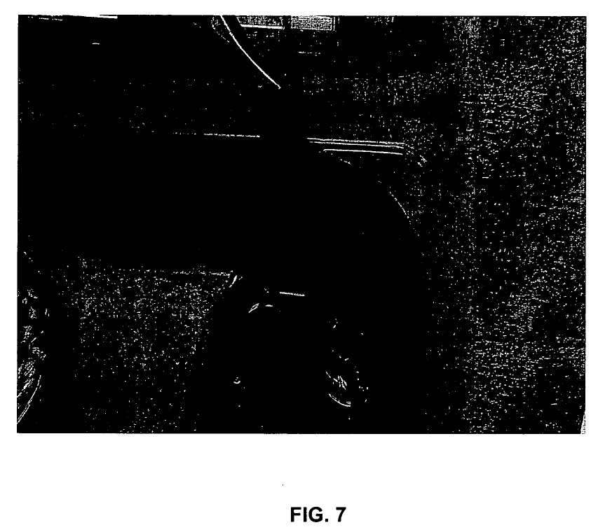

According to yet another embodiment of the present invention, and as illustrated in FIG. 6, a graft 60 is first soaked in a PRP 15 and then inserted into the tibial tunnel. During soaking, the graft takes up about 25% of the PRP. Alternatively, and in accordance with a preferred embodiment shown in FIG. 7, graft 60 may be soaked in the mixture 10 of PRP and hyaluronic acid (FIG. 1). The addition of hyaluronic acid allows the PRP to become more viscous and to adhere better to the graft. In yet another embodiment, the soaked graft may be inserted in the tibial tunnel and a coagulated PRP (such as the coagulated PRP 20 of FIG. 2) is inserted into the tibial tunnel and around the soaked graft, by injection through a portal, for example. Subsequent to the injection of the PRP 50 through the portal and into the tibial tunnel, a cannulated interference screw or retroscrew is plugged, to prevent leaking of the PRP from the target tunnel.

By providing the viscous and/or coagulated PRP within the tibial tunnel, healing of the operative site and bone growth are accelerated. The fixation devices (i.e., interference screws or retroscrews) prevent leakage of the viscous and/or coagulated PRP that promotes the growth of bone and/or tissue structure and the overall healing process.

Optionally, the viscous and/or coagulated PRP may comprise additional lubricants and/or an antiseptic chemical and/or an antibiotic. In this case, other solution excipients such as buffer salts, sugars, anti-oxidants and preservatives to maintain the bioactivity of the PRP and a proper pH of the plasma may be also employed. The additional lubricants and/or the antiseptic and/or the antibiotic will typically be present in the plasma in a predetermined concentration range, which will be dependent upon the particular bone site and application, as well as the specific activity of the antiseptic and/or the antibiotic.

Although the above embodiments have been described above with reference to the viscous and/or coagulated PRP provided at a particular tissue repair site, such as a tibial tunnel, the invention is not limited to this exemplary embodiment. Accordingly, the present invention has applicability to the injection of viscous and/or coagulated platelet-rich plasma and autogenous growth factors to a variety of tunnels and sockets provided within repair sites corresponding to bone, soft tissue or osteochondral tissue, among others.

The above description and drawings illustrate preferred embodiments which achieve the objects, features and advantages of the present invention. It is not intended that the present invention be limited to the illustrated embodiments. Any modification of the present invention which comes within the spirit and scope of the following claims should be considered part of the present invention.

What is claimed is:

1. A method of conducting ligament reconstruction, comprising the steps of:
providing a tunnel within a target tissue;
soaking a graft in a mixture comprising growth factors to form a soaked graft; and
inserting the soaked graft into the tunnel.

2. The method of claim 1, wherein the growth factors are autogenous growth factors.

3. The method of claim 2, wherein the autogenous growth factors are contained in platelet-rich plasma.

4. The method of claim 3, wherein the platelet-rich plasma is obtained by centrifugation of blood.

5. The method of claim 1, further comprising the steps of:
adding a coagulant to the mixture to form a coagulum; and
inserting at least part of the coagulum within the tunnel, subsequent to the step of inserting the soaked graft.

6. The method of claim 5, wherein the coagulant is thrombin or calcium chloride.

7. The method of claim 1, wherein the mixture further comprises hyaluronic acid.

8. The method of claim 1, wherein the mixture further comprises a component selected from the group consisting of lubricants, antiseptics and antibiotics.

9. The method of claim 1, further comprising the step of securing a fixation device at each end of the tunnel.

10. The method of claim 9, wherein the tunnel is a bone tunnel and the fixation device secured at each end of the tunnel is an interference screw.

11. The method of claim 1, wherein the target tissue is bone.

12. The method of claim 1, wherein the target tissue is soft tissue.

13. The method of claim 1, wherein the target tissue is osteochondral tissue.

14. The method of claim 1, wherein the tunnel is a tibial tunnel.

15. A method of promoting tissue growth, comprising the steps of:
forming a bone tunnel within a bone of a patient;
inserting a graft or ligament within the bone tunnel;
injecting a coagulum containing growth factors into the bone tunnel containing the graft or ligament; and
securing both ends of the graft or ligament to the bone tunnel with fixation devices.

16. The method of claim 15, wherein the growth factors are obtained by centrifugation of blood from the patient.

17. The method of claim 15, wherein the coagulum further comprises a component selected from the group consisting of lubricants, antiseptics and antibiotics.

18. The method of claim 15, wherein the coagulum further comprises hyaluronic acid.

19. The method of claim 15, wherein the coagulum is formed with a coagulant comprising thrombin or calcium chloride.

20. The method of claim 15, wherein the fixation devices are cannulated interference screws.
